(19) United States
(12) Patent Application Publication
(10) Pub. No.: US 2022/0265168 A1
(43) Pub. Date: Aug. 25, 2022

(54) REAL-TIME LIMB MOTION TRACKING

(71) Applicant: SAMSUNG ELECTRONICS CO., LTD., Gyeonggi-Do (KR)

(72) Inventors: Wenchuan Wei, Sunnyvale, CA (US); Keiko Kurita, Menlo Park, CA (US); Jilong Kuang, San Jose, CA (US); Jun Gao, Menlo Park, CA (US)

(21) Appl. No.: 17/362,732

(22) Filed: Jun. 29, 2021

Related U.S. Application Data
(60) Provisional application No. 63/152,436, filed on Feb. 23, 2021.

(51) Int. Cl. A61B 5/11; A61B 5/00

(52) U.S. Cl. CPC A61B 5/1122; A61B 5/1116; A61B 5/6815; A61B 5/6824; A61B 5/6823; A61B 5/7267; A61B 2560/0223

(57) ABSTRACT

Limb motion tracking using a single sensor can include capturing acceleration data. The acceleration data can be captured in real time by an IMU sensor of a wearable device worn on a limb of a user. Orientation data can be captured in real time by the IMU sensor concurrently with the acceleration data. Estimated positions of joints of the limb can be determined based on the acceleration data and the orientation data, the joints positions estimated using a machine learning model and relative to a coordinate system. Motion of the limb can be tracked based on the estimated positions determined at different times as the user moves the limb.

### CROSS-REFERENCE TO RELATED APPLICATIONS

This application claims the benefit of U.S. Provisional Patent Application No. 63/152,436 filed on Feb. 23, 2021, which is incorporated herein by reference in its entirety.

### TECHNICAL FIELD

This disclosure relates to sensor-based motion capture, and more particularly, to tracking human limb motions using a single sensor.

### BACKGROUND

Techniques for tracking human limb motion are of increasing interest in academic circles, industry, and various other fields of endeavor. In connection with treating certain health conditions, for example, monitoring a patient's limb motion while the patient performs arm or leg exercises is important in physical therapy to improve the patient's range of limb motion, strength, and flexibility. As a communication tool in human computer interaction, for example, a user's arm motion or gestures are widely used as a communication tool in human computer interaction. These are only two examples of areas in which tracking human limb motion is important. Tracking human limb motion is increasingly important in other areas as well.

### SUMMARY

In one or more embodiments, a method includes capturing acceleration data. The acceleration data is captured in real time by an IMU sensor of a wearable device worn on a limb of a user. The method includes capturing orientation data in real time by the IMU sensor concurrently with the acceleration data. The method includes determining estimated positions of joints of the limb based on the acceleration data and the orientation data. The joint positions are estimated using a machine learning model and are relative to a coordinate system. The method includes tracking motion of the limb based on the estimated positions determined at different times as the user moves the limb.

In one or more embodiments, a system includes a processor configured to initiate operations. The operations include capturing acceleration data. The acceleration data is captured in real time by an IMU sensor of a wearable device worn on a limb of a user. The operations include capturing orientation data in real time by the IMU sensor concurrently with the acceleration data. The operations include determining estimated positions of joints of the limb based on the acceleration data and the orientation data. The joint positions are estimated using a machine learning model and are relative to a coordinate system. The operations include tracking motion of the limb based on the estimated positions determined at different times as the user moves the limb.

In one or more embodiments, a computer program product includes one or more computer readable storage media having instructions stored thereon. The instructions are executable by a processor to initiate operations. The operations include capturing acceleration data. The acceleration data is captured in real time by an IMU sensor of a wearable device worn on a limb of a user. The operations include capturing orientation data in real time by the IMU sensor concurrently with the acceleration data. The operations include determining estimated positions of joints of the limb based on the acceleration data and the orientation data. The joint positions are estimated using a machine learning model and are relative to a coordinate system. The operations include tracking motion of the limb based on the estimated positions determined at different times as the user moves the limb.

This Summary section is provided merely to introduce certain concepts and not to identify any key or essential features of the claimed subject matter. Other features of the inventive arrangements will be apparent from the accompanying drawings and from the following detailed description.

### BRIEF DESCRIPTION OF THE DRAWINGS

The inventive arrangements are illustrated by way of example in the accompanying drawings. The drawings, however, should not be construed to be limiting of the inventive arrangements to only the particular implementations shown. Various aspects and advantages will become apparent upon review of the following detailed description and upon reference to the drawings.

FIGS. 1A and 1B illustrate an example limb motion tracking system.

FIG. 2 illustrates certain operative aspects of a novel neural network implemented with the example system of FIGS. 1A and 1B.

FIGS. 3A-3C illustrate distinct coordinate systems used in joint position translations performed as part of a user orientation identification by the example system of FIGS. 1A and 1B.

FIGS. 4A-4C illustrate distinct calibration postures used for user orientation identification performed by the example system of FIGS. 1A and 1B.

FIG. 5 illustrates an example skeletal hierarchy used for a user orientation identification performed by the example system of FIGS. 1A and 1B.

FIG. 6 illustrates aspects of torso motion tracking optionally performed by the example system of FIGS. 1A and 1B.

FIG. 7 is a flowchart of an example method of limb motion tracking.

FIG. 8 depicts an example portable device in which can be implemented the example system of FIGS. 1A and 1B.

### DETAILED DESCRIPTION

While the disclosure concludes with claims defining novel features, it is believed that the various features described within this disclosure will be better understood from a consideration of the description in conjunction with the drawings. The process(es), machine(s), manufacture(s) and any variations thereof described herein are provided for purposes of illustration. Specific structural and functional details described within this disclosure are not to be interpreted as limiting, but merely as a basis for the claims and as a representative basis for teaching one skilled in the art to variously employ the features described in virtually any appropriately detailed structure. Further, the terms and phrases used within this disclosure are not intended to be limiting, but rather to provide an understandable description of the features described.

This disclosure relates to sensor-based motion capture, and more particularly, to tracking human limb motions using a single sensor. As already noted, tracking human limb motion is of increasing importance in varied fields of endeavor. These fields include, for example, physical therapy wherein a patient performs arm or leg exercises to improve the patient's range of limb motion, strength, and flexibility, handwriting recognition based on hand and wrist movement, and human-computer interactions based on human gestures. Notwithstanding the wide interest in tracking human limb motion, there yet remain significant challenges to accurate, efficient, and comfortable limb motion tracking. In the specific context of physical therapy, for example, tracking limb motion accurately as a patient performs arm or leg exercises typically cannot be monitored without the supervision of a professional physical therapist. Some studies have shown, for example, that unsupervised home-based exercise programs for patients can produce poor outcomes for patients with certain health conditions such as Parkinson's disease. More generally, even for healthy individuals engaged in normal exercises activity, incorrect limb motion can lead to suboptimal results or even injuries if performed without the assistance of an experienced physical trainer.

Different motion capture systems have been developed for tracking human limb motion. Camera-based systems use computer vision techniques to track a user's 3D motion from video images captured by one or more cameras. Camera-based approaches, however, can raise privacy concerns and other issues. Moreover, the accuracy of camera-based approaches can be adversely affected by occlusion. The quality of video images can be affected by factors such as poor resolution, distance between the user and the camera(s), and the like. Wearable devices that use multiple inertial measurement unit (IMU) sensors to track the user's motion are an alternative to camera-based approaches. Tracking a user's limb motion with a wearable device, however, typically requires that the device include at least two sensors to perform the tracking, one on the upper extremity of a limb, and the other on the limb's lower extremity. The need for multiple sensors typically increases complexity and may be an annoyance to the user.

An aspect of the systems, methods, and computer program products disclosed herein is limb motion tracking with enhanced accuracy while using only a single sensor.

In accordance with inventive arrangements described herein, example methods, systems, and computer program disclosed track limb motion based on estimated positions of the joints of a user's limb (e.g., elbow and writ joints, hip and knee joints). The estimates are generated in real time based on real-time acceleration and orientation data generated by a single IMU sensor in response to limb movements of the user. The joint positions estimated by tracking the limb motion in real time are expressed in a user coordinate system. For example, in tracking arm motion, the elbow position is represented by points on a virtual sphere centered at the shoulder of the user and the position of the wrist by points on a virtual sphere centered at the user's elbow.

An aspect of limb tracking in accordance with inventive arrangements disclosed is the training and use of machine learning models for estimating joint positions, which in certain arrangements is a recurrent neural network (RNN). Generally, the RNN is characterized by use of nonlinear processing units and as such is a nonlinear dynamic system. Unlike neural networks in which the state of network is determined by a current input, an RNN is configured to account for initial and past states and perform serial processing. The RNN of the inventive arrangements disclosed in contrast to other neural networks can thus model sequences of data such that each sample can be assumed to be dependent on previous ones. In this way, the RNN processes and preserves temporal information.

Compared with other machine learning and statistic learning algorithms that also process time series, the RNN more accurately performs real-time estimations or predictions. For example, although a Hidden Markov Model (HMI) uses dynamic programming to find an optimal state sequence from the input sequence, the HMM works only after an entire input sequence is obtained and is computationally expensive. Certain studies have shown that an HMM-based approach can cause as much as a 9-minute delay for a 1-minute trajectory time and thus can only be applied offline, not in real time. The RNN of the inventive arrangements disclosed overcomes these and other limitations.

Additional aspects of the example methods, systems, and computer program disclosed herein is user orientation identification for identifying a specific user's orientation in tracking and translation of coordinate system positions into a user coordinate system. Other aspects include determining a user-specific calibration posture and skeletal normalization to enhance limb tracking accuracy based on unique physical characteristics and conditions of the user. Feasible points of joint positioning on the spheres of the coordinate system are demarcated by the user's range of motion (ROM) and skeletal size (e.g., length of forearm). The ROM and skeletal size can be specific to the user and can be determined by the example methods, systems, and computer program disclosed. These aspects further reduce the uncertainty typically associated with estimation of limb joint positions based on IMU sensor data.

Further aspects of the embodiments described within this disclosure are described in greater detail with reference to the figures below. For purposes of simplicity and clarity of illustration, elements shown in the figures have not necessarily been drawn to scale. For example, the dimensions of some of the elements may be exaggerated relative to other elements for clarity. Further, where considered appropriate, reference numbers are repeated among the figures to indicate corresponding, analogous, or like features.

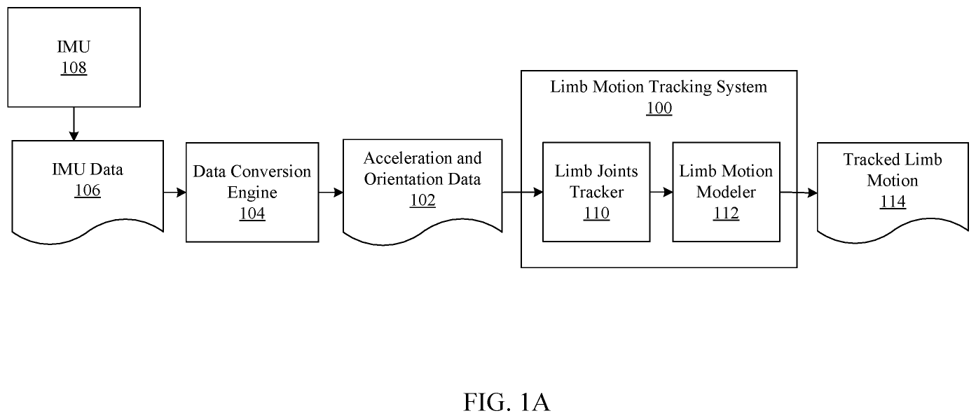

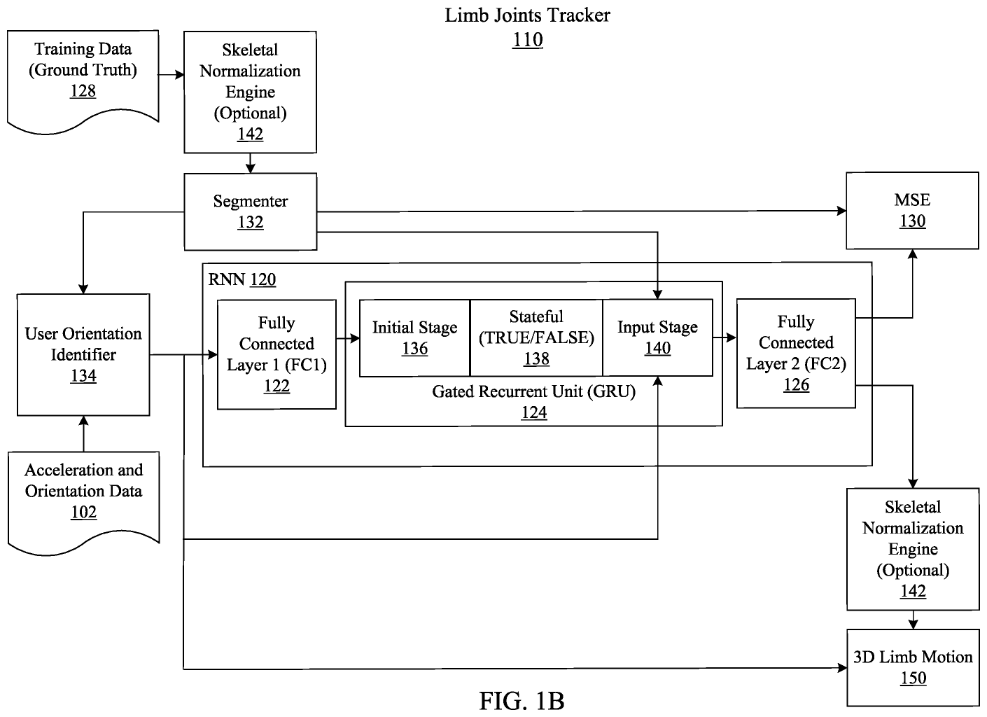

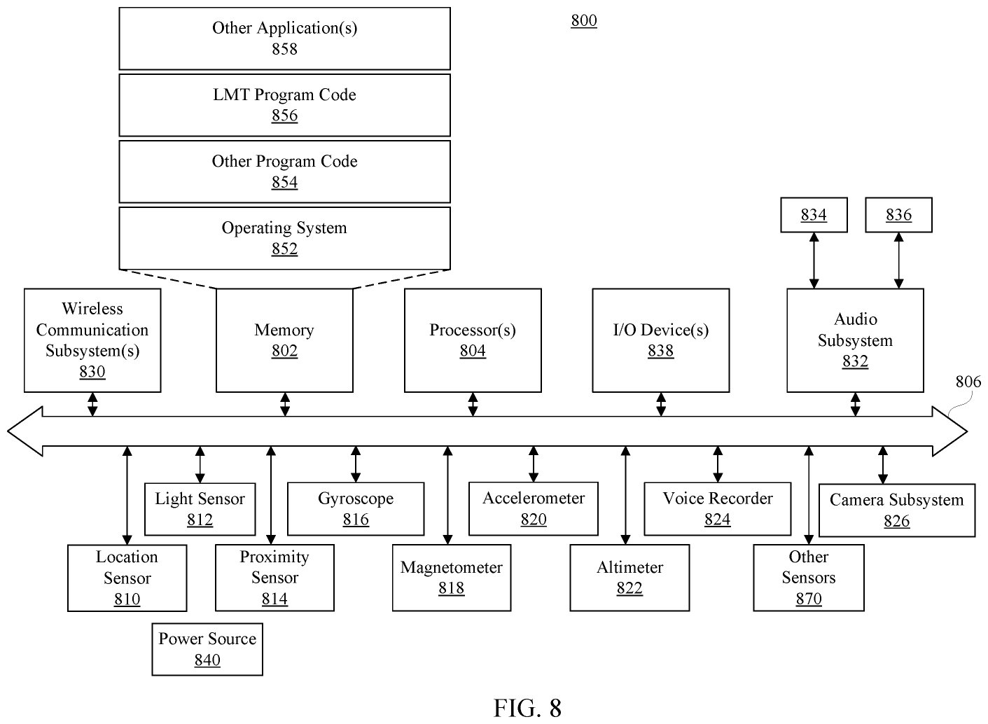

FIGS. 1A and 1B illustrate example limb motion tracking system (system) 100. System 100 can be implemented in machine-readable code executing on a processor and/or in hardwired circuitry of a wearable device (e.g., smartwatch, smart sock) such as device 800 described with reference to FIG. 8. Illustratively, system 100 tracks limb motion in real time based on acceleration and orientation data 102 obtained from a single sensor. Acceleration and orientation data 102 are machine-readable data that is generated by data conversion engine 104, which receives IMU data 106 generated by IMU 108, which is the sole source of sensor data, and which is operatively coupled with or embedded in the wearable device in which system 100 is implemented.

In various arrangements, IMU 108 comprises a 3-axis accelerometer, a 3-axis gyroscope, and a 3-axis magnetometer that sense, respectively, acceleration, angular velocity, and magnetic fields. IMU data 106 thus comprises a combination of raw sensor data based on which data conversion engine 104 generates acceleration and orientation data 102 that is input to system 100. The combination of raw sensor data generated by IMU 108 can be processed by data conversion engine 104 into motion data, including free acceleration, and orientation data. In certain arrangements, the free acceleration and orientation are determined from raw sensor data based on data fusion, a process by which data from several different sensors are fused to compute, for example, the acceleration and orientation of a device in a three-dimensional space. Various sensor fusion algorithms are implemented in software libraries and integrated circuits

System 100, as illustrated in FIG. 1A, comprises limb joints tracker 110 which determines the positions of limb joints (e.g., elbow and wrist, or knee and ankle) in real time based on acceleration and orientation data 102 generated in response to motion of the user's limb. The positions of the limb joints estimated by limb joints tracker 110 are input from limb joints tracker 110 to limb motion modeler 112. Based on the real-time determinations of limb joints estimated by limb joints tracker 110 from acceleration and orientation data 102, limb motion modeler 112 outputs tracked limb motion 114. Tracked limb motion 114, in certain arrangements, is presented to a user as a 3D model of the user's limb motion. The model can be used, for example, to model the user's limb movements during physical therapy or exercise. For example, the model can be used to measure the user's range of motion. The model can be used, for example, for recognizing the user's handwriting, or to recognize gestures for controlling a computer or other device.

FIG. 1B illustrates in greater detail limb joints tracker 110. Limb joints tracker 110 uses a trained machine learning model to estimate the positions of limb joints (e.g., elbow and wrist) based on acceleration and orientation data 102. The machine learning model, in various arrangements, is RNN 120. RNN 120 illustratively comprises three layers. The first layer is fully connected 1 (FC1) layer 122. The second layer is gated recurrent unit (GRU) layer 124. And the third layer is fully connected 2 (FC2) layer 126.

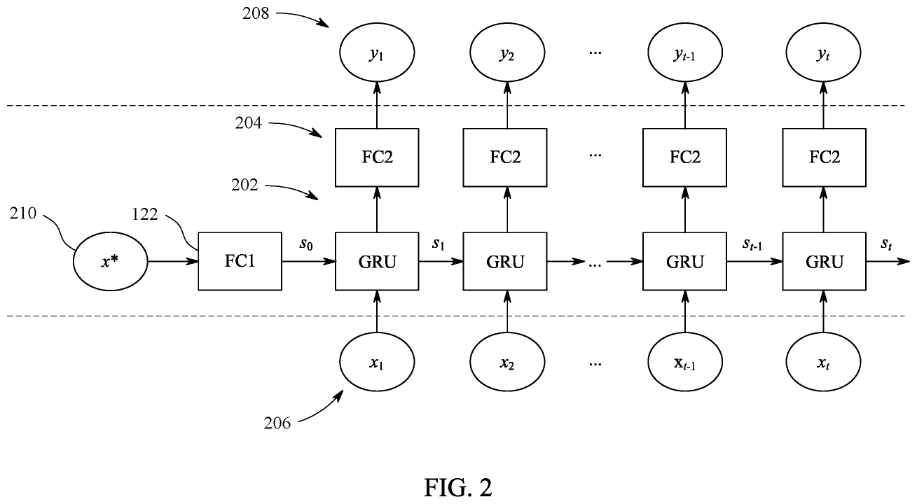

Referring additionally to FIG. 2, certain operative aspects of RNN 120 are schematically illustrated in the processing of temporal information for estimating current, time-specific positions of the limb joints. A time-based sequence of data is processed with RNN 120 providing virtual network architecture 200, which illustratively comprises multiple frames at times 1 through t generated by the distinct layers of RNN 120. Row 202 depicts GRU layer 124 at time frames 1 through t. Row 204 depicts FC1 layer at time frame 0. Row 206 depicts at each time frame 1 through t, inputs x₁, . . . , xₜ₋₁, xₜ. Each input comprises time-based acceleration and orientation data, which is input to GRU layer 124, whose output is in turn fed to FC2 layer 126 at each time frame. Row 208 depicts real-time outputs inputs y₁, . . . , yₜ₋₁, yₜ at each of the respective time frames l through t. Each real-time output comprises a time-specific 3D position of a user's limb joints (e.g., elbow and wrist, hip and knee) with respect to a predefined user coordinate system (described in detail below). GRU layer 124 at each time frame 1 through t−1 feeds the previous frame's state Sₜ₋₁ to the current frame at t so that RNN 120 learns to make estimations for the current frame at t from available historical data.

GRU layer 124 differs from conventional GRU models whose initial state, by default, is set to zero. Rather than the initial zero-filled initial state of conventional GRU models, GRU layer 124 learns the initial state of the model from initial position and velocity data 210. Initial position and velocity data 210 is input to FC1 layer 122, which is trained with machine learning to convert initial position and velocity data 210 to an initial state, S₀. Initial state S₀ is input to GRU layer 124. Providing the initial state S₀ to GRU layer 124 as determined by converting initial position and velocity data 210 with trained FC1 layer 122 enables limb joints tracker 110 to accurately estimate real-time limb joint positions.

Referring still to FIGS. 1A and 1B, RNN 120 is trained using training data 128, a set of feature vectors whose elements are joint positions of a limb known to correctly estimate limb joint positions. Training data 128 establishes the ground truth for assessing the accuracy of RNN 120. The training data is input to RNN 120, and the estimates generated by RNN 120 are compared with the ground truth of training data 128. In certain arrangements, the accuracy is assessed based on mean square error (MSE) 130 of the estimated positions with respect to the true positions of the ground truth.

Training data 128 comprises IMU acceleration and orientation data, which can be segmented and shuffled by segmenter 132. Prior to segmentation and shuffling, training data 128 optionally can be processed by user orientation and identifier 134 to calibrate the limb joints tracker 110 to the physicality of the specific user, as described in detail below. For each data segment generated by segmenter 132, the initial joint position velocities are sent to FC1 layer 122 and the output used to set the initial state of GRU layer 124, as described above. Because the training data 128 is segmented and the segments shuffled, FC1 layer 122 is used to convert the initial joint positions and velocities into the initial stage 136 of the GRU layer 124. GRU layer 124 is stateless (stateful 138 is set to FALSE) during the training phase. Therefore, the final state of each segment of training data 128 at each time frame of the training sequence is not used as the initial state of the next frame in the training sequence. For each training sequence, the initial state is learned from the ground-truth initial joint positions and velocities through the FC1 layer 122, whose output is fed to initial stage 136 of GRU layer 124.

Training data 128, comprising acceleration and orientation data, is input to input 140 of GRU layer 124, and the output of GRU layer 124 is fed to FC2 layer 126 which in turn outputs the final estimations of the joint positions that are compared with the ground truth to determine MSE 130, which reflects the accuracy of the model after training. Training can be repeated iteratively until an acceptable level of accuracy measured by MSE 130 is achieved.

At runtime, the initial joint positions and velocities can be pre-defined by the user calibration posture described below and conveyed to FC1 layer 122. The output of FC1 layer 122 is used to set the initial state of the GRU layer 124. The initial state is a one-time setting. Moreover, the initial state of the GRU layer 124 can be determined based on a user-specific calibration posture selected by the user prior to the real-time tracking.

During real-time tracking, acceleration and orientation data 102 generated from IMU data 106 captured by IMU 108 are input to GRU layer 124 after user orientation identification performed by user orientation identifier 134. Distinct from the training stage, at runtime GRU layer 124 is set as stateful (stateful 138 is set to TRUE) so that each at each time frame the internal hidden state is passed to the next time frame. FC2 layer 126 outputs the real-time estimates of joint positions. Optionally, as described below, the joint positions can be estimated by RNN 120 trained based on a pre-defined normalized skeleton corresponding to the specific user and determined by optional skeletal normalization engine 142, and the outputs of estimated real-time joint positions resealed by optional skeletal rescaler 148. In the context of tracking the user's arm motion, for example, the arm motion can be fully characterized by the joint positions and by the forearm pronation-supination rotation, which are obtained from the orientation data captured by an IMU sensor of a device (e.g., smartwatch) worn on the user's wrist.

In certain arrangements, limb joints tracker 110 acceleration and orientation data is initially processed by user orientation identification system 134 based on a personalized calibration posture. User orientation identification system 134 provides a user-specific calibration based on the unique physical attributes of the user, such as the limb length and ordinary orientation or posture of the user for enhancing the accuracy with which the user's limb motion is tracked. The calibration performed by user orientation identification system 134 allows the tracking results of the limb motion by limb joints tracker 110 to be represented in a unique user coordinate system Sᵤₛₑᵣ. The joint positions within user coordinate system Sᵤₛₑᵣ can be determined by translating positions in different coordinate systems. User orientation identification system 134 can perform translations between distinct coordinate systems. Three coordinate systems are local earth coordinate system Sₑₐᵣₜₕ, sensor coordinate system Sₛₑₙₛₒᵣ, and user coordinate system Sᵤₛₑᵣ.

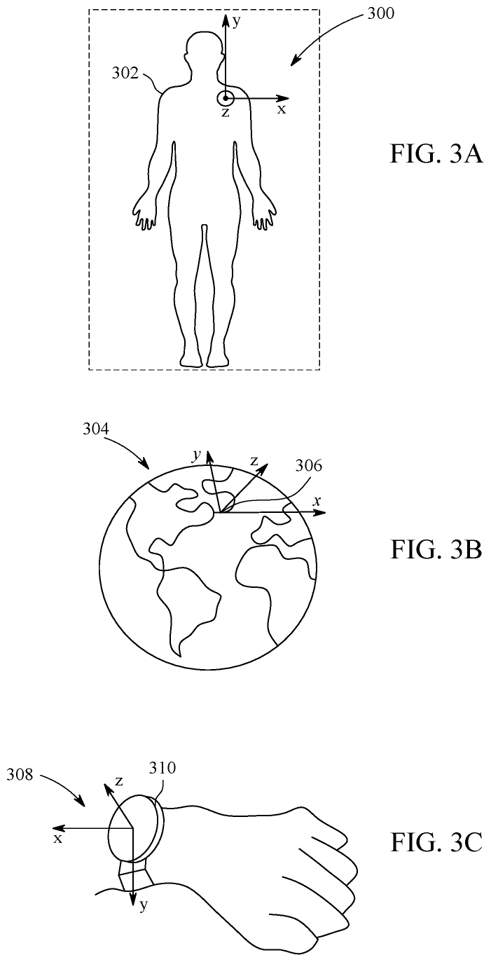

FIGS. 3A-3C illustrate the three coordinate systems. FIG. 3A depicts example user coordinate system Sᵤₛₑᵣ 300 with respect to user 302. The origin of user coordinate system Sᵤₛₑᵣ 300 is located at user 302's shoulder. The x-axis extends leftward from the origin, the y-axis upward from the origin, and the z-axis outward from the plane. FIG. 3B depicts local earth coordinate system Sₑₐᵣₜₕ 304 centered at earth location 306. The x-axis extends eastward from the origin at earth location 306, the y-axis northward from the origin, and the z-axis upward from the origin. FIG. 3C depicts example sensor coordinate system Sₛₑₙₛₒᵣ 308, the sensor embedded in smartwatch 310 worn on user 302's wrist. Illustratively, the x-axis extends outward from the origin centered on the face of smartwatch 310, with the y-axis perpendicular to the x-axis, and the z-axis perpendicular to both the x-axis and y-axis. For the final tracking results of the limb motion determined by limb joints tracker 110 to be presented in user coordinate system Sᵤₛₑᵣ, user orientation identification system 134 performs joint position translations between the distinct coordinate systems. The translations are based on user-specific calibration postures.

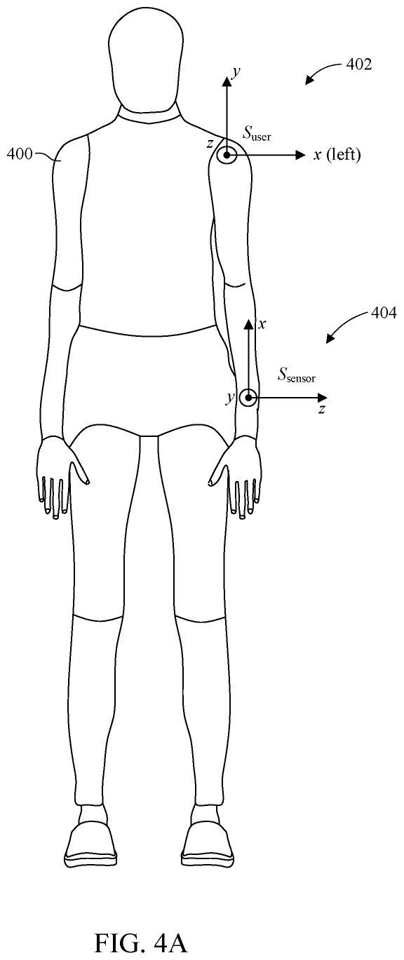

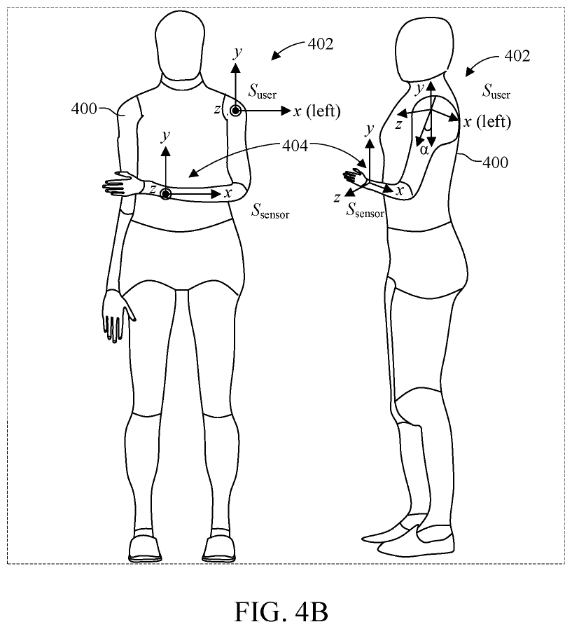

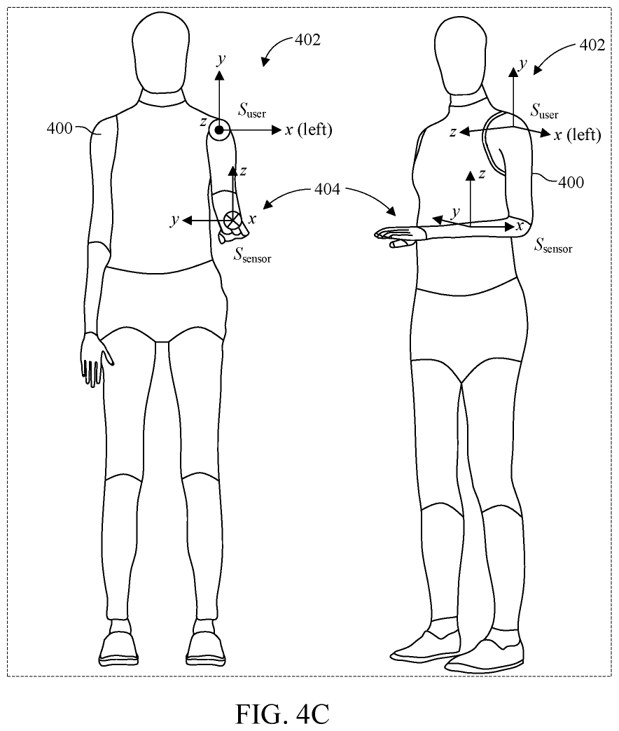

FIGS. 4A-4C depict three example calibration postures corresponding to different physical conditions of user 400. Illustratively, the example calibration postures pertain specifically to arm motion tracking, but the same procedures apply equally with respect to leg motion tracking, for example. User coordinate system Sᵤₛₑᵣ 402 is centered at user 400's shoulder, and sensor coordinate system Sₛₑₙₛₒᵣ 404 is centered at user 400's wrist. Sensor coordinate system Sₛₑₙₛₒᵣ 404 can be centered with a sensor embedded in a device (e.g., smartwatch) worn on user 400's wrists. User 400 can select from among predetermined calibration postures or define a new one based on the user's specific health conditions or personal preferences. To perform user orientation identification, the user stays stationary in selected posture briefly (e.g., approximately 3 seconds) before motion tracking is initiated.

FIG. 4A depicts a default calibration posture. User 400's arm is perpendicular to the floor, the elbow is straight, and the palm faces user 400's body. The rotation matrix, Rₛₑₙₛₒᵣ₋ₜₒ₋ᵤₛₑᵣ, transforms the positions in the coordinates of sensor coordinate system Sₛₑₙₛₒᵣ to positions in user coordinate system Sᵤₛₑᵣ. For the default calibration posture, the rotation matrix, Rₛₑₙₛₒᵣ₋ₜₒ₋ᵤₛₑᵣ, is:

$${R}_{sensor - t o - u s e r} = [ \begin{matrix}0 & 0 & 1 \\ 1 & 0 & 0 \\ 0 & 1 & 0\end{matrix} ] .$$

The elbow position, p₀^{elbow} of user 400 in the calibration posture is (0, −lᵤ, 0), where lᵤ is the length of user 400's upper arm, and l_{f} is the length of user 400's forearm. User 400's wrist position p₀^{wrist} is (0, −(lᵤ+l_{f}),0)

The FIG. 4B illustrates a front view (left) and side view (right) of a calibration posture for user 400 assuming user 400's elbow has only a limited range of motion. In the calibration position, user 400's left forearm is extended rightward, parallel with the floor and against user 400's body. User 400's upper arm rotates forward with angle α. The rotation matrix, Rₛₑₙₛₒᵣ₋ₜₒ₋ᵤₛₑᵣ, for transforming the positions in the coordinates of sensor coordinate system Sₛₑₙₛₒᵣ to positions in user coordinate system Sᵤₛₑᵣ is:

$${R}_{sensor - t o - u s e r} = [ \begin{matrix}1 & 0 & 0 \\ 0 & 0 & 1 \\ 0 & - 1 & 0\end{matrix} ] .$$

The elbow position, p₀^{elbow} of user 400 in this calibration posture is (0, −lᵤ·cos α, lᵤ·sin α), where lᵤ is the length of user 400's upper arm, l_{f} is the length of user 400's forearm, and user 400's upper arm rotates forward with angle α. User 400's wrist position p₀^{wrist} is (−l_{f}, −lᵤ·cos α, lᵤ·sin α).

FIG. 4C illustrates a front view (left) and side view (right) of a calibration posture for user 400 assuming user 400 suffers impaired mobility of both the elbow and shoulder. In the calibration position, user 400 points the left forearm forward, parallel with the floor. User 400's palm faces downward. User 400's upper arm is perpendicular to the floor. The rotation matrix, Rₛₑₙₛₒᵣ₋ₜₒ₋ᵤₛₑᵣ, for transforming the joint positions in the coordinates of sensor coordinate system Sₛₑₙₛₒᵣ to positions in user coordinate system Sᵤₛₑᵣ is:

$${R}_{s e n s o r - t o - u s e r} = [ \begin{matrix}0 & - 1 & 0 \\ 0 & 0 & 1 \\ - 1 & 0 & 0\end{matrix} ] .$$

The elbow position, p₀^{elbow} of user 400 in this calibration posture is (0, −lᵤ, 0), where lᵤ is the length of user 400's upper arm. User 400's wrist position p₀^{wrist} is (0, −lᵤ, l_{f}).

As described above, the initial joint velocities and positions are pre-defined with respect to the calibration posture at runtime. The initial joint velocities are zero given that a user remains stationary while performing the calibration procedure. The user's initial joint positions p₀^{elbow} and p₀^{wrist} can also be determined from the user-selected calibration postures.

The default calibration posture (FIG. 4A) is one typically used, the user straightening the arms and keeping the elbow extended at 180 degrees. If, however, the user requires physical therapy and has impaired mobility of the arm (e.g., owing to an elbow injury), the user may not be able to extend the elbow to 180 degrees. When a user cannot correctly perform the calibration posture or as instructed, the R_{sensorToUser}, does not likely reflect a true user posture, which can lead to incorrect user calibration results and affect the accuracy of joint tracking. To address the problem, the user can select among alternative calibration postures, two of which are described above, choosing the one that best accommodates the user's specific condition. As also described, the user can define a personalized calibration posture. Optionally, in certain arrangements, a visual presentation of an avatar is presented to the user with a device display (not shown) and the user can define the personalized calibration posture by rotating the virtual bones of the avatar, and the corresponding features (R_{sensorToUser}, p₀^{elbow}, p₀^{wrist}) can be calculated from the user-specified posture.

In the context of arm motion tracking, a user with limited elbow range of motion can select the calibration posture corresponding to limited elbow range of motion (FIG. 4B), which requires an elbow angle of rotation of only about 90 degrees. When performing the posture, the user will naturally rotate the shoulder and the upper arm to place the forearm in front of and against the user's body, though doing so may be challenging for a user suffering from a shoulder injury. Accordingly, for a user suffering from a shoulder injury the user can opt for the other calibration posture (FIG. 4C), which does not require shoulder rotation.

As noted above, the orientation of the sensor output is in the sensor coordinate system Sₛₑₙₛₒᵣ with respect to local earth coordinate system Sₑₐᵣₜₕ. Therefore, an average sensor orientation can be calculated with the user maintaining the calibration posture as the rotation between Sₑₐᵣₜₕ and Sₛₑₙₛₒᵣ, that is, R_{earthToSensor}.

Given the above results, the transformation between Sᵤₛₑᵣ and Sₑₐᵣₜₕ, that is R_{earthToSensor}, can be calculated from the cross product of the two matrices R_{sensorToUser} and R_{earthToSensor}:

R_{earthToUser}=R_{sensorToUser}×R_{earthToSensor}

R_{earthToUser} represents the user's orientation with respect to the local earth coordinate system, that is, the direction the user is facing. As the proposed tracking system is used only when the user does not move the torso or change orientation, the transformation between Sᵤₛₑᵣ and Sₑₐᵣₜₕ is fixed during the tracking. The IMU orientation and acceleration data can be transformed to Sᵤₛₑᵣ as follows:

Accelᵤₛₑᵣ=R_{earthToUser}×Accelₑₐᵣₜₕ, and

Orientᵤₛₑᵣ=R_{earthToUser}×Orientₑₐᵣₜₕ,

where Accelₑₐᵣₜₕ and Orientₑₐᵣₜₕ are the acceleration and orientation data in the original local earth coordinate system Sₑₐᵣₜₕ, and where Accelᵤₛₑᵣ and Orientᵤₛₑᵣ are the acceleration and orientation data in the user coordinate system Sᵤₛₑᵣ.

Limb joints tracker 110's estimates of joint positions of a user are influenced not only by the user-specific calibration posture, but also by the user's body size. The different body sizes of different users thus affect the estimated joint positions of users even though the users assume the same calibration posture initially for training RNN 120. Accordingly, in certain arrangements, limb joints tracker 110 optionally includes skeletal normalization unit 142, which incorporates the body size of the user into the ground truth represented by training data 128. Incorporation of the body size for training RNN 120 makes the model more robust to variations in body sizes. Skeletal normalization unit 142 normalizes the ground-truth of the joint positions during training of RNN 120. The normalization can be based on a skeletal hierarchy.

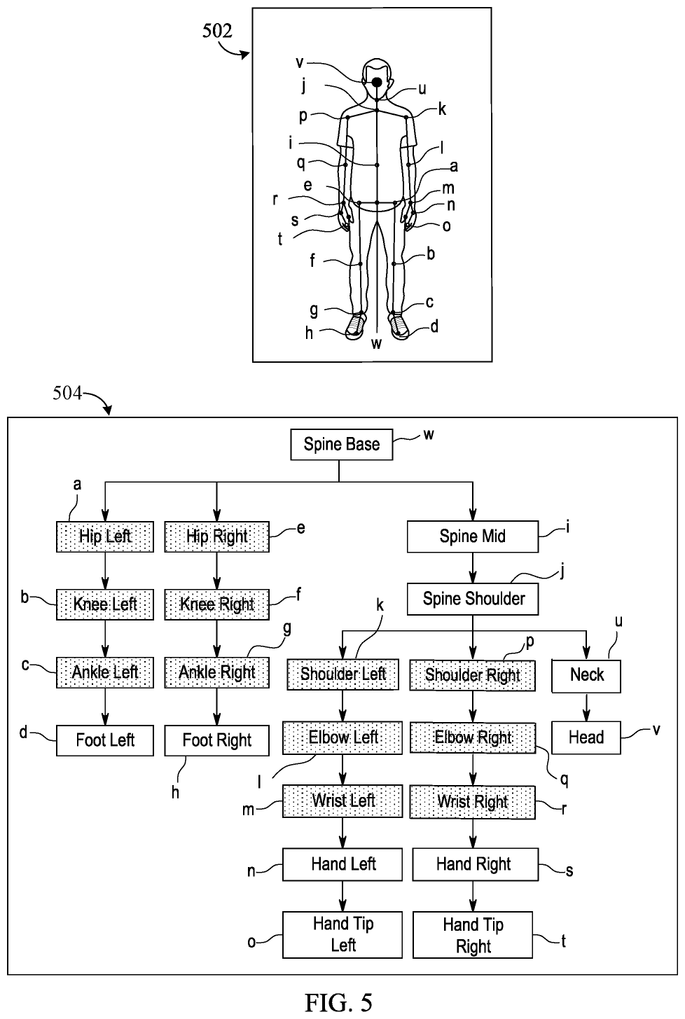

FIG. 5 illustrates example skeletal hierarchy 500. Skeletal hierarchy 500 enumerates the individual joints of normalized user skeleton 502. The individual joints are then arranged in hierarchy 504. Normalized values (e.g., length of the upper arm=0.3 m, length of forearm=0.28 m) can be assigned to each element of hierarchy 504. The position of each joint is normalized starting with joints higher in hierarchy 504 using the following calculation:

$${p}_{t h i s} = {p}_{parent} + {l}_{normalized} \cdot \frac{T {P}_{t h i s} - T {P}_{p a r e n t}}{‖ {TP}_{t h i s} - T {P}_{p a r e n t} ‖}$$

where TPₜₕᵢₛ and TPₚₐᵣₑₙₜ are, respectively, the ground-truth positions of a joint and its parent joint. pₜₕᵢₛ and pₚₐᵣₑₙₜ are the normalized positions of this joint and its parent joint, respectively, in normalized user skeleton. RNN 120 is trained with data culled from the same normalized user skeleton and according to the described normalization process applied with respect to the specific user. At runtime, the joint position estimates are also based on the normalized skeleton. The estimates can be rescaled by skeletal rescaler 148 to match the specific size of the user's body. The rescaled estimates are used to generate 3D limb motion 150, a real-time 3D rendering of the limb motion of the user.

The typical manner of collecting data to train an RNN model is collecting N sequences of equal length r (herein, “recording” indicates collecting one sequence from a user). With system 100, however, because the user orientation identification phase calculates the transformation between the coordinate systems, subjects from whom training data is acquired must repeat the user orientation identification process for each recording. Therefore, this data collection method is time consuming and laborious. To address this problem, system 100 optionally utilizes a novel data collection method. System 100 obtains only 5 to 8 recordings from each subject. Each subject changes body orientation and performs the user orientation identification for each recording. Instead of recording a sequence of length r, however, system 100 collects a much longer sequence of length L (L≈50r) for each recording. System 100 segments the long sequence into multiple segments of length r, each segment with a 50% overlap of the adjacent segments. In this manner, system 100 acquires approximately L/(50% r)≈100 sequences from each long recording, which is much more convenient and effective than collecting 100 recordings from a subject separately.

With this novel data augmentation approach, however, system 100 cannot use the default zero-filled initial state for RNN 120 as the initial joint positions and velocities of the segmented sequences are different. The initial position and velocity, though, can be critical in determining the real-time joint positions. Different initial positions and/or velocities can lead to different joint positions even with the same real-time input from an IMU sensor. System 100 remedies this with FC1 layer 122, which as described converts the initial joint position and velocity data to the initial state of GRU layer 124. For each segmented sequence, the initial joint positions are the joint positions in the first frame of the segment. The initial joint velocity can be calculated as

v=[p(1)−p(0)]·fₛ,

where p(0) and p(1) are joint positions in the first and second frame, and fₛ is the sampling frequency.

In training the RNN 120, the timesteps of the training sequence r can be preset (e.g., set as 300 each such that each training sequence includes 300 frames, which is approximately 5 seconds as the sampling frequency of the IMU sensor is 60 Hz). At runtime, the timesteps are set to 1. The IMU data can be sent to RNN 120 frame by frame to enable real-time tracking.

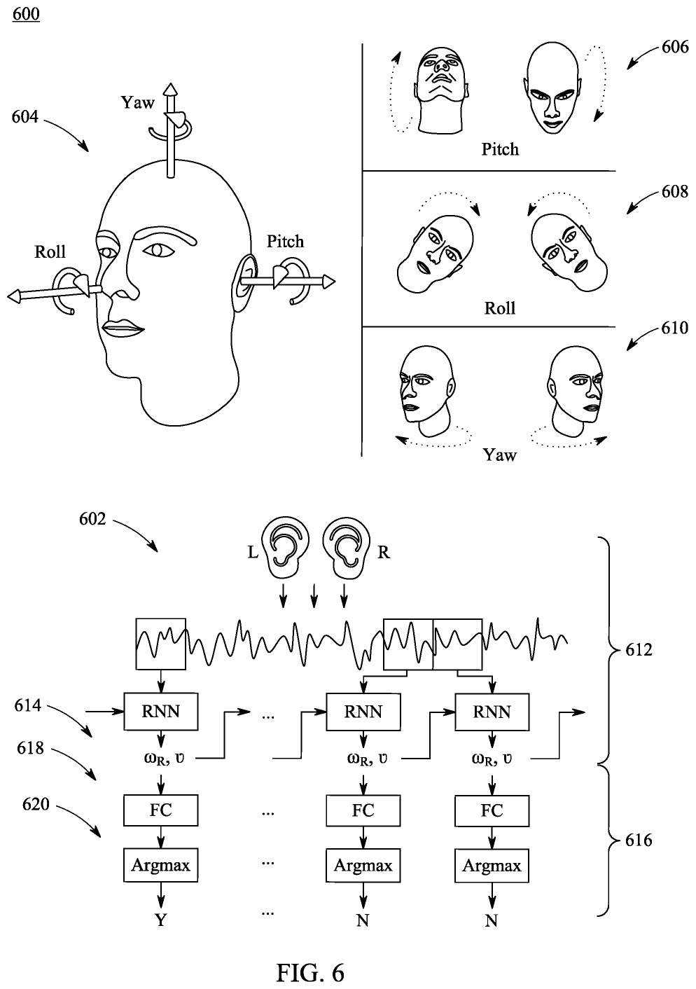

FIG. 6 depicts example detection system 600, which is configured to detect torso movements of a user during the limb joint tracking. Torso movements during tracking can cause incorrect tracking results. To date, providing limb motion tracking using a single sensor that allows torso motion has proved elusive. For example, with a wrist-mounted IMU sensor for detecting when the user's wrist is moving forward, there are two possible scenarios: 1) the user's entire body is moving forward but the user's arm is stationary relative to the user's torso; or 2) only the user's wrist is moving forward while the user's torso remains static. With conventional tracking it is difficult to distinguish between the two scenarios. To overcome the problem, system 100 optionally includes detection system 600, which detects the user's torso motion based on the user's head motion using the IMU sensors of a pair of earbuds 602.

Firstly, the user's head motion can be classified into three distinct categories. The first is head rotation 604, which includes pitch 606, roll 608, and yaw 610. The second is head motion caused by user torso motion, in which the user's head remains stationary relative to the user's torso, but the user's torso is moving (e.g., due to the user walking). The third is mixed motion in which the user's torso is moving while the user's head is also moving.

Detection system 600 is configured to distinguish between head motion caused by torso motion and pure head rotation. Detection system 600 comprises two parts. The first is RNN model 612, which is trained using machine learning to estimate a user's head rotation and displacement. When the user wears earbuds 602, the IMU sensors embedded therein continuously capture the acceleration and orientation data. When the user's head remains stationary relative to the user's torso, but the user's torso is moving, the movement causes changes in the IMU data generated with earbuds 602. Detection system 600 uses non-overlapping sliding windows of 1 second duration to segment the IMU data. Each data segment is sent to RNN model 612 to estimate the head rotation, represented by the angular velocity ω_{R}, and displacement, represented by the velocity v, 614. The second part of detection system 600, is classifier 616, which classifies whether detected motion is pure head rotation or exclusively torso motion. Head rotation and displacement 614 are sent to a FC layer 618, followed by Argmax layer 620, which outputs the final classification results.

RNN model 612 and classifier 616 are trained separately. The ground-truth head rotation and displacement data can be obtained from other sensors (e.g., cameras). The ground-truth torso motion can be correctly labelled manually. RNN model 612 utilizes temporal information to improve the classification accuracy. For example, if a user had torso motion in a previous data segment and the head motion velocity v is high, it is highly likely that the torso is still moving in the current segment.

Detection system 600 provides a reliable torso motion detection model, which is able to detect whether and when the user moves the torso during joint tracking. An RNN-based joints tracking model is used only when no torso motion is detected. When detection system 600 detects that the user moves the torso, the joint tracking is paused and real-time notification (e.g., haptic, visual, or audio feedback) is conveyed to the user to remind the user not to move the torso. The user can perform a brief (e.g., 3-second) user orientation identification before the real-time recording.

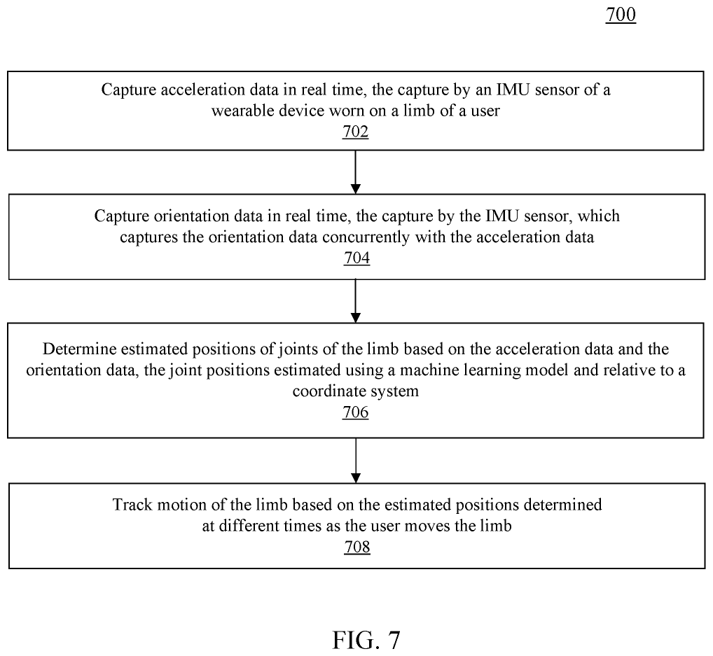

FIG. 7 is a flowchart of example method 700 for limb motion tracking. Method 700 can be performed by a system such as the systems described with respect to FIGS. 1-6. At block 702, the system captures acceleration data. The system captures the acceleration data in real time with an IMU sensor of a wearable device worn on a limb of a user. The system, at block 704, captures orientation data. The system captures the orientation data in real time with the IMU sensor. The system captures the orientation data concurrently with the acceleration data.

At block 706, the system determines estimated positions of joints of the limb based on the acceleration data and the orientation data. The estimated positions are determined using a machine learning model and are relative to a coordinate system. The system tracks the motion of the limb based on the estimated positions determined at different times as the user moves the limb.

The machine learning model can be an RNN that includes an initial, fully connect layer. The initial, fully connected layer can be trained to convert initial positions of the joints and initial velocity of the joints into an initial state of a gated recurrent unit of the RNN.

In certain arrangements, the system translates the estimated positions from the coordinate system to a user coordinate system. The system can present results of the tracking to the user in the user coordinate system. The system can translate the coordinate system to a user-specific coordinate system based on a user calibration. The user calibration posture can be a personalized calibration posture determined by the system based on a calibration procedure performed by the user in advance of the tracking. The user calibration posture can be a personalized calibration posture that the system determines based in part on a health condition of the user.

In other arrangements, the system also can perform a skeletal normalization specific to the user. The skeletal normalization performed by the system can be based on user measurements for establishing a ground truth.

In still other arrangements, the system can detect torso movements of the user. The system can detect the torso movements from data generated by sensors of a second device operatively coupled to a wearable device worn by the user. The second device, in certain arrangements, can comprise a pair of earbuds worn by the user.

FIG. 8 illustrates an example portable device 800 in accordance with one or more embodiments described within this disclosure. Portable device 800 can include a memory 802, one or more processors 804 (e.g., image processors, digital signal processors, data processors), and interface circuitry 806.

In one aspect, memory 802, processor(s) 804, and/or interface circuitry 806 are implemented as separate components. In another aspect, memory 802, processor(s) 804, and/or interface circuitry 806 are integrated in one or more integrated circuits. The various components of portable device 800 can be coupled, for example, by one or more communication buses or signal lines (e.g., interconnects and/or wires). In one aspect, memory 802 can be coupled to interface circuitry 806 via a memory interface (not shown).

Sensors, devices, subsystems, and/or input/output (I/O) devices can be coupled to interface circuitry 806 to facilitate the functions and/or operations described herein, including the generation of sensor data. The various sensors, devices, subsystems, and/or I/O devices can be coupled to interface circuitry 806 directly or through one or more intervening I/O controllers (not shown).

For example, location sensor 810, light sensor 812, and proximity sensor 814 can be coupled to interface circuitry 806 to facilitate orientation, lighting, and proximity functions, respectively, of portable device 800. Location sensor 810 (e.g., a GPS receiver and/or processor) can be connected to interface circuitry 806 to provide geo-positioning sensor data.

Portable device 800 can include an IMU comprising gyroscope 816, magnetometer 818, and accelerometer 820. Magnetometer 818 can be connected to interface circuitry 806 to provide sensor data that can be used to determine the direction of magnetic North for purposes of directional navigation. Accelerometer 820 can be connected to interface circuitry 806 to provide sensor data that can be used to determine change of speed and direction of movement of a device in three dimensions.

Altimeter 822 (e.g., an integrated circuit) can be connected to interface circuitry 806 to provide sensor data that can be used to determine altitude. Voice recorder 824 can be connected to interface circuitry 806 to store recorded utterances. Camera subsystem 826 can be coupled to an optical sensor 828. Optical sensor 828 can be implemented using any of a variety of technologies. Examples of optical sensor 828 include a charged coupled device (CCD), a complementary metal-oxide semiconductor (CMOS) optical sensor, and the like. Camera subsystem 826 and optical sensor 828 can be used to facilitate camera functions, such as recording images and/or video clips (hereafter “image data”). In one aspect, image data is a subset of sensor data.

Communication functions can be facilitated through one or more wireless communication subsystems 830. Wireless communications subsystem(s) 830 can include radio frequency receivers and transmitters, optical (e.g., infrared) receivers and transmitters, and so forth. The specific design and implementation of wireless communication subsystem(s) 830 can depend on the specific type of portable device 800 implemented and/or the communication network(s) over which portable device 800 is intended to operate.

For purposes of illustration, wireless communication subsystem(s) 830 can be designed to operate over one or more mobile networks (e.g., GSM, GPRS, EDGE), a Wi-Fi network that can include a WiMax network, a short-range wireless network (e.g., a Bluetooth network), and/or any combination of the foregoing. Wireless communication subsystem(s) 830 can implement hosting protocols such that portable device 800 can be configured as a base station for other wireless devices.

Audio subsystem 832 can be coupled to a speaker 834 and a microphone 836 to facilitate voice-enabled functions, such as voice recognition, voice replication, digital recording, audio processing, and telephony functions. Audio subsystem 832 is able to generate audio type sensor data. In one or more embodiments, microphone 836 can be utilized as a respirator sensor.

I/O devices 838 can be coupled to interface circuitry 806. Examples of I/O devices 838 include, for example, display devices, touch-sensitive display devices, track pads, keyboards, pointing devices, communication ports (e.g., USB ports), network adapters, buttons or other physical controls, and so forth. A touch-sensitive device such as a display screen and/or a pad is configured to detect contact, movement, breaks in contact, and the like using any of a variety of touch sensitivity technologies. Example touch-sensitive technologies include, for example, capacitive, resistive, infrared, and surface acoustic wave technologies, other proximity sensor arrays or other elements for determining one or more points of contact with a touch-sensitive device, and the like. One or more of I/O devices 838 can be adapted to control functions of sensors, subsystems, and such of portable device 800.

Portable device 800 further includes a power source 840. Power source 840 able to provide electrical power to various elements of portable device 800. In one embodiment, power source 840 is implemented as one or more batteries. The batteries can be implemented using any of a variety of different battery technologies, whether disposable (e.g., replaceable) or rechargeable. In another embodiment, power source 840 is configured to obtain electrical power from an external source and provide power (e.g., DC power) to the elements of portable device 800. In the case of a rechargeable battery, power source 840 further can include circuitry that is able to charge the battery or batteries when coupled to an external power source.

Memory 802 can include random access memory (e.g., volatile memory) and/or non-volatile memory, such as one or more magnetic disk storage devices, one or more optical storage devices, flash memory, and so forth. Memory 802 can store operating system 852, such as LINUX, UNIX, a mobile operating system, an embedded operating system, and the like. Operating system 852 can include instructions for handling system services and for performing hardware-dependent tasks.

Memory 802 can store additional program code 854. Examples of other program code 854 can include instructions to facilitate communicating with one or more additional devices, one or more computers, and/or one or more servers; graphic user interface processing; processing instructions to facilitate sensor-related functions; phone-related functions; electronic messaging-related functions; Web browsing-related functions; media processing-related functions; GPS and navigation-related functions; security functions; camera-related functions, including Web camera and/or Web video functions; and so forth.

Memory 802 include limb motion tracking (LMT) program code 856. LMT program code 856 can implement a limb motion tracking system (e.g., system 100). Memory 802 can also store one or more other applications 862.

The various types of instructions and/or program code described are provided for purposes of illustration and not limitation. The program code can be implemented as separate software programs, procedures, or modules. Memory 802 can include additional instructions or fewer instructions. Moreover, various functions of portable device 800 can be implemented in hardware and/or software, including in one or more signal processing and/or application-specific integrated circuits.

Program code stored within memory 802 and any data used, generated, and/or operated on by portable device 800 are functional data structures that impart functionality to a device when employed as part of the device. Further examples of functional data structures include, for example, sensor data, data obtained via user input, data obtained via querying external data sources, baseline information, and so forth. The term “data structure” refers to a physical implementation of a data model's organization of data within a physical memory. As such, a data structure is formed of specific electrical or magnetic structural elements within a memory. A data structure imposes physical organization on the data stored in the memory that is used by a processor.

In certain embodiments, one or more of the various sensors and/or subsystems described with reference to portable device 800 can be separate devices that are coupled or communicatively linked to portable device 800 through wired or wireless connections. For example, one or more (or all) of location sensor 810, light sensor 812, proximity sensor 814, gyroscope 816, magnetometer 818, accelerometer 820, altimeter 822, voice recorder 824, camera subsystem 826, audio subsystem 832, and so forth can be implemented as separate systems or subsystems that operatively couple to portable device 800 by way of I/O devices 838 and/or wireless communication subsystem(s) 830.

Portable device 800 can include fewer components than those shown or include additional components other than those shown in FIG. 8 depending on the specific type of system that is implemented. Additionally, the particular operating system and/or application(s) and/or other program code included can also vary according to system type. Moreover, one or more of the illustrative components can be incorporated into, or otherwise form a portion of, another component. For example, a processor can include at least some memory.

Portable device 800 is provided for purposes of illustration and not limitation. A device and/or system configured to perform the operations described herein can have a different architecture than illustrated in FIG. 8. The architecture can be a simplified version of portable device 800 and can include a processor and memory storing instructions. The architecture can include one or more sensors as described herein. Portable device 800, or a similar system, can collect data using the various sensors of the device or sensors coupled thereto. It should be appreciated, however, that portable device 800 can include fewer sensors or other additional sensors. With this disclosure, data generated by a sensor is referred to as “sensor data.”

Example implementations of portable device 800 include, for example, a smartphone or other mobile device or phone, a wearable computing device (e.g., smartwatch), a dedicated medical device or other suitable handheld, wearable, or comfortably carriable electronic device, capable of sensing and processing sensor-detected signals and data. It will be appreciated that embodiments can be deployed as a standalone device or deployed as multiple devices in a distributed client-server networked system. For example, in certain embodiments, a smartwatch can operatively couple to a mobile device (e.g., smartphone). The mobile device may or may not be configured to interact with a remote server and/or computer system.

The terminology used herein is for the purpose of describing particular embodiments only and is not intended to be limiting. Notwithstanding, several definitions that apply throughout this document now will be presented.

As defined herein, the singular forms “a,” “an,” and “the” include the plural forms as well, unless the context clearly indicates otherwise.

As defined herein, “another” means at least a second or more.

As defined herein, “at least one,” “one or more,” and “and/or,” are open-ended expressions that are both conjunctive and disjunctive in operation unless explicitly stated otherwise. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together.

As defined herein, “automatically” means without user intervention.

As defined herein, “includes,” “including,” “comprises,” and/or “comprising,” specify the presence of stated features, integers, steps, operations, elements, and/or components, but do not preclude the presence or addition of one or more other features, integers, steps, operations, elements, components, and/or groups thereof.

As defined herein, “if” means “in response to” or “responsive to,” depending upon the context. Thus, the phrase “if it is determined” may be construed to mean “in response to determining” or “responsive to determining” depending on the context. Likewise the phrase “if [a stated condition or event] is detected” may be construed to mean “upon detecting [the stated condition or event]” or “in response to detecting [the stated condition or event]” or “responsive to detecting [the stated condition or event]” depending on the context.

As defined herein, “one embodiment,” “an embodiment,” “in one or more embodiments,” “in particular embodiments,” or similar language mean that a particular feature, structure, or characteristic described in connection with the embodiment is included in at least one embodiment described within this disclosure. Thus, appearances of the aforementioned phrases and/or similar language throughout this disclosure may, but do not necessarily, all refer to the same embodiment.

As defined herein, the phrases “in response to” and “responsive to” mean responding or reacting readily to an action or event. Thus, if a second action is performed “in response to” or “responsive to” a first action, there is a causal relationship between an occurrence of the first action and an occurrence of the second action. The phrases “in response to” and “responsive to” indicate the causal relationship.

As defined herein, “real time” means a level of processing responsiveness that a user or system senses as sufficiently immediate for a particular process or determination to be made, or that enables the processor to keep up with some external process.

As defined herein, “substantially” means that the recited characteristic, parameter, or value need not be achieved exactly, but that deviations or variations, including for example, tolerances, measurement error, measurement accuracy limitations, and other factors known to those of skill in the art, may occur in amounts that do not preclude the effect the characteristic was intended to provide.

As defined herein, the terms “user,” “individual,” “patient,” and “subject” each refer to a human being.

The terms first, second, etc. may be used herein to describe various elements. These elements should not be limited by these terms, as these terms are only used to distinguish one element from another unless stated otherwise or the context clearly indicates otherwise.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
capturing acceleration data, wherein the acceleration is data captured in real time by an IMU sensor of a wearable device worn on a limb of a user;
capturing orientation data, wherein the orientation data is captured in real time by the IMU sensor concurrently with the acceleration data;
determining, with computing hardware, estimated positions of joints of the limb based on the acceleration data and the orientation data, wherein the positions are estimated using a machine learning model and are relative to a coordinate system; and
tracking, with the computing hardware, motion of the limb based on the estimated positions determined at different times as the user moves the limb.

2. The method of claim 1, wherein the machine learning model is a recurrent neural network (RNN) that includes an initial, fully connect layer trained to convert initial positions of the joints and initial velocity of the joints into an initial state of a gated recurrent unit of the RNN.

3. The method of claim 1, further comprising translating the estimated positions from the coordinate system to a user coordinate system and presenting results of the tracking to the user in the user coordinate system.

4. The method of claim 3, wherein the translating translates the coordinate system to a user-specific coordinate system based on a user calibration.

5. The method of claim 4, wherein the user calibration corresponds to a personalized calibration posture determined based on a calibration procedure performed by the user in advance of the tracking.

6. The method of claim 4, wherein the user calibration corresponds to a personalized calibration posture determined in part by a health condition of the user.

7. The method of claim 1, further comprising performing skeletal normalization based on user measurements for establishing a ground truth.

8. The method of claim 1, further comprising detecting torso movement of the user, wherein the torso movement is detected by sensors of a second device operatively coupled to the wearable device.

9. The method of claim 8, wherein the second device is a pair of earbuds worn by the user.

10. A system, comprising:
a processor configured to initiate operations including:
capturing acceleration data, wherein the acceleration data is captured in real time by an IMU sensor of a wearable device worn on a limb of a user;
capturing orientation data, wherein the orientation data is captured in real time by the IMU sensor concurrently with the acceleration data;
determining estimated positions of joints of the limb based on the acceleration data and the orientation data, wherein the positions are estimated using a machine learning model and are relative to a coordinate system; and
tracking, with the computing hardware, motion of the limb based on the estimated positions determined at different times as the user moves the limb.

11. The system of claim 10, wherein the machine learning model is a recurrent neural network (RNN) that includes an initial, fully connect layer trained to convert initial positions of the joints and initial velocity of the joints into an initial state of a gated recurrent unit of the RNN.

12. The system of claim 10, wherein the processor is configured to initiate further operations including translating the estimated positions from the coordinate system to a user coordinate system and presenting results of the tracking to the user in the user coordinate system.

13. The system of claim 12, wherein the translating translates the coordinate system to a user-specific coordinate system based on a user calibration.

14. The system of claim 13, wherein the user calibration corresponds to a personalized calibration posture determined based on a calibration procedure performed by the user in advance of the tracking.

15. The system of claim 13, wherein the user calibration corresponds to a personalized calibration posture determined in part by a health condition of the user.

16. The system of claim 10, wherein the processor is configured to initiate further operations including performing skeletal normalization based on user measurements for establishing a ground truth.

17. The system of claim 10, wherein the processor is configured to initiate further operations including detecting torso movement of the user, wherein the torso movement is detected by sensors of a second device operatively coupled to the wearable device.

18. The system of claim 17, wherein the second device is a pair of earbuds worn by the user.

19. A computer program product, the computer program product comprising:
one or more computer-readable storage media and program instructions collectively stored on the one or more computer-readable storage media, the program instructions executable by a processor to cause the processor to initiate operations including:
capturing acceleration data, wherein the acceleration data is captured in real time by an IMU sensor of a wearable device worn on a limb of a user;
capturing orientation data, wherein the orientation data is captured in real time by the IMU sensor concurrently with the acceleration data;
determining estimated positions of joints of the limb based on the acceleration data and the orientation data, wherein the positions are estimated using a machine learning model and are relative to a coordinate system; and
tracking, with the computing hardware, motion of the limb based on the estimated positions determined at different times as the user moves the limb.

20. The computer program product of claim of claim 19, wherein the machine learning model is a recurrent neural network (RNN) that includes an initial, fully connect layer trained to convert initial positions of the joints and initial velocity of the joints into an initial state of a gated recurrent unit of the RNN.
